(19) United States
(12) Patent Application Publication
(10) Pub. No.: US 2023/0190203 A1
(43) Pub. Date: Jun. 22, 2023

(54) HUMAN HEALTH RISK ASSESSMENT METHOD

(71) Applicant: OBSHCHESTVO S OGRANICHENNOJ OTVETSTVENNOSTYU "PARMA-TELEKOM", Perm (RU)

(72) Inventors: Eduard Gennadievich NELYUBIN, Perm (RU); Tatyana Ivanovna PROKOPENKO, Perm (RU); Sergej Alekseevich SINAJSKIJ, Moscow (RU); Dmitry Vladimirovich TACHKIN, Perm (RU); Leonid Ivanovich TIHOMIROV, Perm (RU)

(73) Assignee: OBSHCHESTVO S OGRANICHENNOJ OTVETSTVENNOSTYU "PARMA-TELEKOM", Perm (RU)

(21) Appl. No.: 17/995,829

(22) PCT Filed: Mar. 31, 2021
(86) PCT No.: PCT/RU2021/050087

§ 371 Date: Oct. 7, 2022

(30) Foreign Application Priority Data
Apr. 9, 2020 (RU) 2020113220

(51) Int. Cl. A61B 5/00; G16H 50/30

(52) U.S. Cl. CPC A61B 5/7246; G16H 50/30; A61B 5/7275; A61B 5/681

(57) ABSTRACT

The invention relates to systems for diagnosing human condition obtained by a personal device worn by a subject. The technical effect is a greater versatility in assessing risks, and a greater reliability and efficiency of health risk assessment. According to the invention, a series of templates are preliminarily prepared, including a set of interrelated critical parameter values and temporal characteristics thereof. Signals are received from at least one wearable personal device, each of the received signals is converted into a binary signal, wherein the signal is given a value of “1” if the signal exceeds a threshold of a critical parameter value which is stored in one of the plurality of pre-prepared templates, and a value of “0” if not. The binary signals are then compared with each other and, if the values of “1” temporally coincide among the set of signals, a decision is made about the presence of certain health risks.

### TECHNICAL FIELD

The invention relates to systems for diagnosing the human conditions based on measured functional parameters obtained from human wearable personal devices.

### BACKGROUND ART

The prior art knows various methods useful for assessing the human health condition on the basis of signals coming from various sensors.

Thus, the prior art knows a method of monitoring of normal or abnormal physiological events in patients by analyzing their biomedical signals, according to the international application W0200357025, published on 24 Dec. 2003, IPC A61B 05/00. A biomedical signal to be analyzed is investigated in the following manner. First, an unprocessed signal, e.g. electrocardiography signal using a corresponding electrode, is obtained. Second, adaptive segmentation of this signal is performed. Further, some features are retrieved from such unprocessed signal. Next, clustering of temporal and waveform features of the signal is performed. Finally, based on the data obtained, medical interpretation of the clusters is done.

Patent EP2156788, published on 24 Feb. 2010, IPC A61B 05/00, discloses a method of measuring vital signs in a time series. Vital parameters are continuously measured by the vital indicator measurement module. The vital indicator measurement module determines whether a person can drive a vehicle basing on a medical condition.

The method known from the prior art, which is the closest to the inventive method claimed in the present application, is a method of detecting pathological fluctuations in physiological signals for diagnosing human diseases, as described in the invention patent application US20100234748, published on 16 Sep. 2010, IPC A61B 05/04.

The known method includes performing a sliding window analysis to find sequences in the physiological signal data that correspond to amplitude- and duration-corrected versions of the template function within a specified tolerance.

The known method includes the following steps:
receiving physiological signal time series data;
obtaining a template for time series data;
selecting the template function that corresponds to the template data of the time series;
analyzing time series data to compare sequences in the time series data to a template function, where one or more sequences contain fluctuations;
calculating one or more oscillation characteristics based on the analysis;
identifying the risk of a clinical condition associated with one or more characteristics.

### DISCLOSURE OF THE INVENTION

Technical effect to be achieved due to the present invention is increasing the versatility of risk assessment, reliability and efficiency, due to the ability to work with signals from different types of sensors and signals of different types of functional parameters.

The method for assessing human health risk includes the following operations.

First, a series of templates are preliminarily prepared, each template including a set of interrelated critical parameter values and temporal characteristics thereof in terms of duration and periodicity, signals containing measured functional parameters are received from at least one wearable device, each of the received signals is converted into a binary signal at a given time interval, wherein the signal is given a value of “1” if the signal exceeds a threshold of a critical parameter value which is stored in one of the plurality of pre-prepared templates, and a value of “0” if not.

The binary signals are then compared with each other and, if the values of “1” temporally coincide among the set of signals of each of the pre-prepared template, a decision is made about the presence of certain health risks.

Improving the versatility of risk assessment in the claimed method is provided by the entire set of features of the claimed invention.

The step of preliminarily preparing a series of templates, each template including a set of interrelated critical parameter values and temporal characteristics thereof in terms of duration and periodicity allows to link various functional parameters characterizing a particular critical health factor into a single template. The choice of critical parameter values and their temporal characteristics in terms of duration and periodicity is based on verified medical data.

Further, signals containing measured functional parameters from wearable personal devices in real time are converted into a binary signal, «1» [symbol] «0», by comparing these signals with the critical value of the parameter of each of the previously prepared templates.

This allows heterogeneous signals from wearable devices to be converted into a single form, with each signal carrying the information that the critical value of this parameter is not exceeded—«0», or is exceeded—«1». It is also important to know for how long this signal has been exceeded, or with what periodicity.

Then, within a set of signals of each of the created templates, the signals of the binary form of different parameters are compared between themselves, and at a temporal coincidence of values “1” a signal “1” is received at the output of the template for a certain time, with a certain periodicity. The presence of such a value allows to make a decision about the presence of a certain health risk.

In addition, templates are preliminarily prepared for functional parameters received from wearable personal devices.

Each template includes at least two parameters out of the parameters obtained from wearable personal devices.

Signals received from said wearable personal device are signals containing, in particular, the following parameters: heart rate, sleep or wakefulness state, type of human physical activity, energy expenditure and inflow, body hydration state, sleep phases, stress level. Besides, before converting signals from wearable personal devices into binary signals, an average value of the signal from the wearable personal device at a given time interval is determined.

Furthermore, after converting each of the received signals into a binary signal, a single stream is formed from the binary signals.

Apart from the foregoing, when a signal from said wearable personal device exceeds the threshold of a critical parameter value, a value of such excess and a duration of such excess are stored. Taking into consideration a magnitude of the excess and the duration of that excess allows, when deciding whether a risk factor is present or not, to determine more accurately the human health conditions.

In particular, it is possible to obtain from a single signal received from said wearable personal device in the process of converting it into a binary signal as many binary signals of a given parameter as there are different critical values of this parameter in the templates.

Also, one or more time windows are used for each template, with which the incoming data characterizing them for each of the signals is correlated.

Additionally, a length of each time window is determined by a specific template.

Besides, an overall assessment of human health risk is performed based on health risk signals.

### BRIEF DESCRIPTION OF THE DRAWING FIGURES

FIG. 1 shows a general flowchart of method steps.

FIG. 2 shows a flowchart for creating templates.

FIG. 3 shows signal conversion graphs containing the measured functional parameters from a wearable personal device into a binary form signal.

FIG. 4 shows graphs of the results of comparing binary signals within the signal set of each of the prepared templates and graphs of certain health risks.

FIG. 5 shows an example of a scheme of interaction between wearable devices and a health risk assessment system.

FIG. 6 shows example of converting signals containing measured functional parameters from a wearable personal device into a binary signal.

FIG. 7 shows another example of converting signals containing measured functional parameters from a wearable personal device into a binary signal.

### EMBODIMENTS OF THE INVENTION

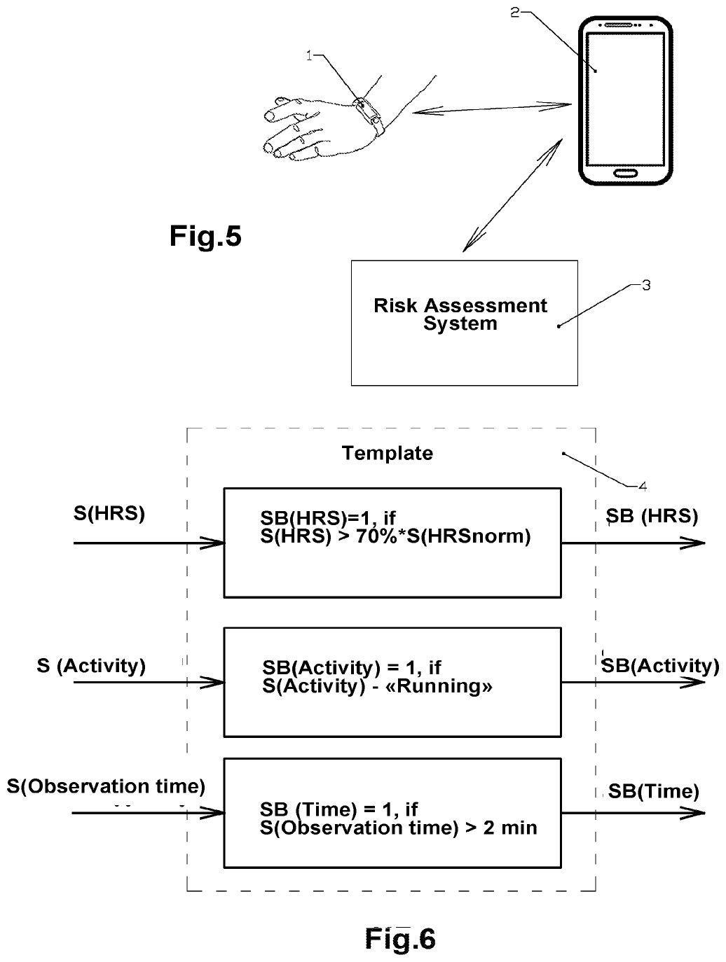

Wearable personal devices 1 are designed primarily to measure functional parameters and to inform the owner of this device about the received parameters (FIG. 5). These devices can also be linked to another wearable device, such as a cell phone 2 or a tablet. At present, these devices do not involve a sufficiently detailed assessment of the risks to human health.

The method of health risk assessment allows to implement a system 3 (FIG. 5) of human health risk assessment by means of information processing tools. For example, cloud computing tools, management and control devices, in particular, a personal account of the user on the web-page of the system or in the smartphone application. Interaction between the elements of such a system can be provided by means of standard means and protocols of data transfer.

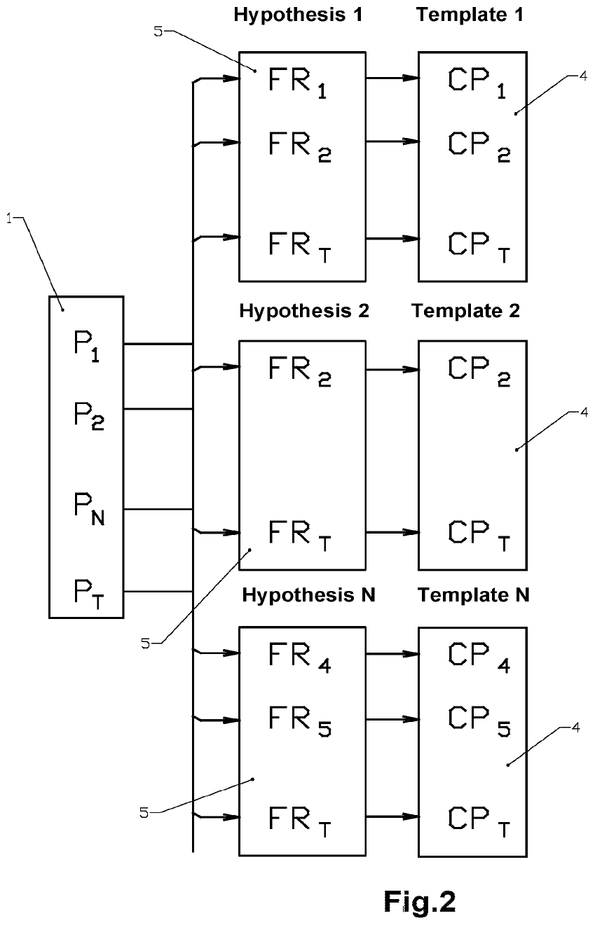

A number of templates 4 are preliminarily prepared (FIG. 2), each of which includes a set of interrelated values of critical parameters and their temporal characteristics in terms of duration and periodicity for signals from wearable personal devices containing measured functional parameters. Such parameters may include: heart rate, sleep or wakefulness state; type of human physical activity, energy expenditure and inflow, body hydration state, sleep phases, stress level.

The procedure for creating template 4 is shown in the flowchart (FIG. 2).

Template 4, which reflects a specific health state, refers to a set of interrelated hypotheses 5 (FIG. 2) set for each of the FR health risk factors that can be identified based on signals S(P) containing measured functional parameters P obtained from a wearable personal device.

In fact, each of the templates 4 reflects a hypothesis about the possible risk to human health when several P parameters are combined. It should be noted that time is also one of the parameters since the temporal characteristics in terms of the duration and periodicity of P parameters should be taken into account when assessing health risks.

When forming hypotheses about a possible risk for human health in case of having several P parameters combined, objective data accumulated by medicine and reflecting cause-and-effect relations between the disease and the preceding pattern of changes in the physiological parameters P of a person are used. On the basis of these data critical CP values of those parameters P are formed, which can be measured by means of a wearable personal device 1.

An example of one such possible templates 4 is shown in FIG. 6. The heart rate signal (HRS) is used as the signal P parameters S (P); the characteristic of the state in which the person is, and this can be the parameters “calm state”, “walking”, “running”, and the parameter “Time”. The value of the critical parameter CP for the parameter signal S (HRS) is defined as «S(HRS)>70%*S(HRS_{NORM})». In other words, If the HRS signal data exceeds the HRS norm by more than 70%, such parameter is considered critical.

The value of the critical parameter CP for the signal of parameter S (Activity), state “Running”.

Critical condition CP for the parameter S time (Observation time) is «>2 min».

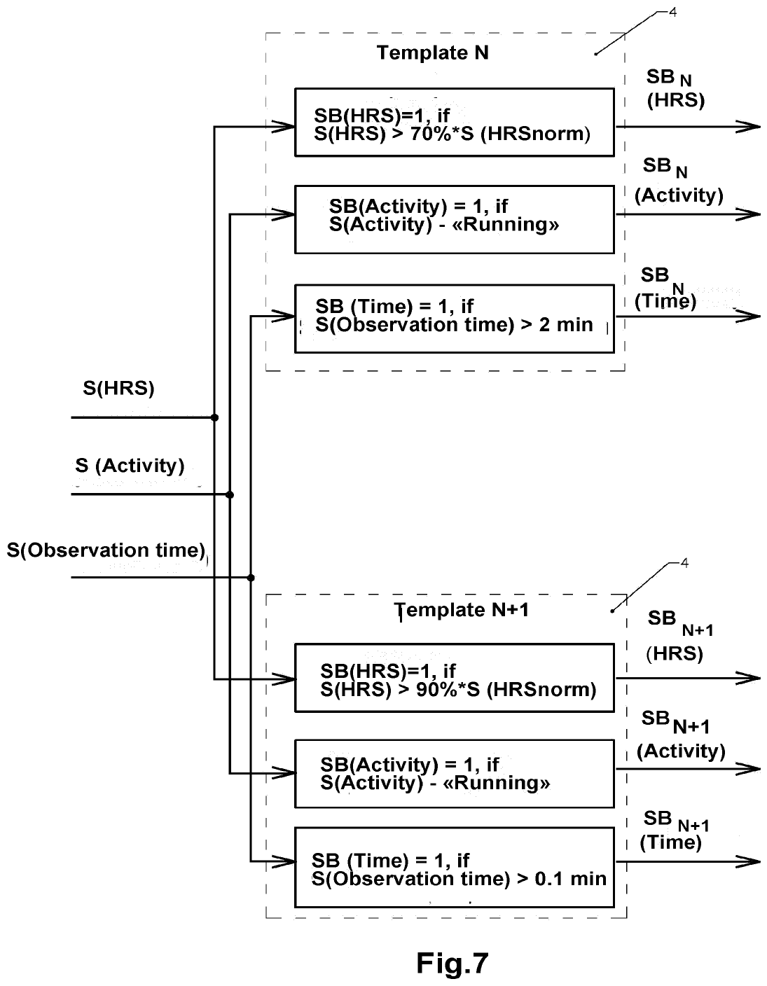

FIG. 7 shows another example in which two templates 4 are formulated based on the same parameters P. Template N is the template from the example in FIG. 6. Template N+1 based on the same parameters P is associated with another hypothesis about a possible risk to human health. This hypothesis assumes the following values of the critical parameters.

The value of the critical parameter CP for the parameter signal S (HRS) is defined as «S(HRS)>90%*S(HRS_{NORM})». In other words, if the HRS signal data exceeds the HRS norm by more than 90%, such parameter is considered critical. Value of the critical parameter CP for the signal of parameter S (Activity—state “Running”).

Critical condition CP for the parameter S time (Observation time) is «>0.1 min».

The example given on FIG. 7 demonstrates that there can be several templates even for the same combination of parameters. The number of templates depends only on understanding what number of risks is possible to determine using the available data from wearable personal devices.

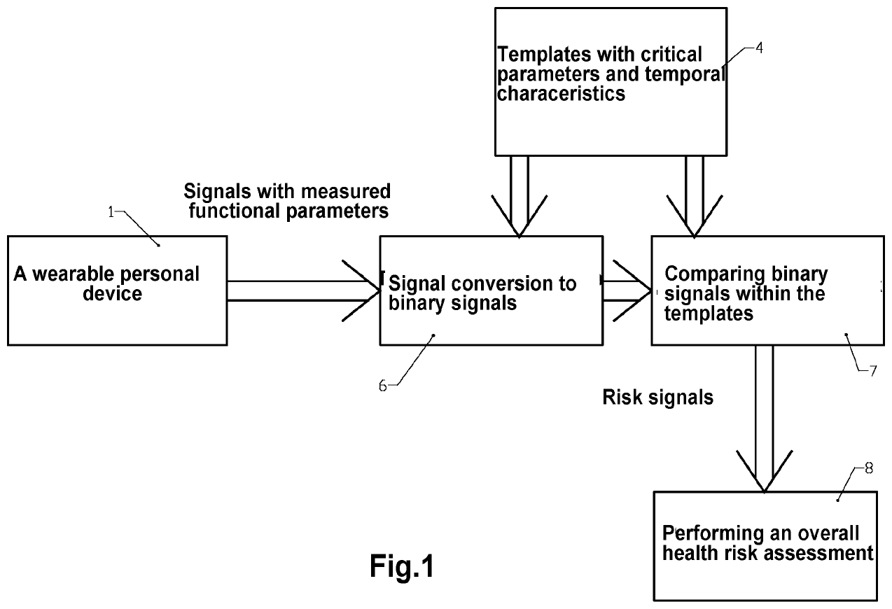

The method of human health risk assessment implemented in Risk Assessment System 3 is performed as follows (FIG. 1).

S(P) signals containing the measured functional parameters P are received from a wearable personal device 1, or from two devices: a wearable device 1 and a mobile phone 2.

Further, signal conversion block 6 is configured to convert each of the received signals into a binary signal at a given time interval. In addition, value «1» is assigned to the signal when this signal exceeds the threshold of the critical value of the parameter stored in one of the set of pre-formed templates 4, and value «0» is assigned to the signal if there is no excess.

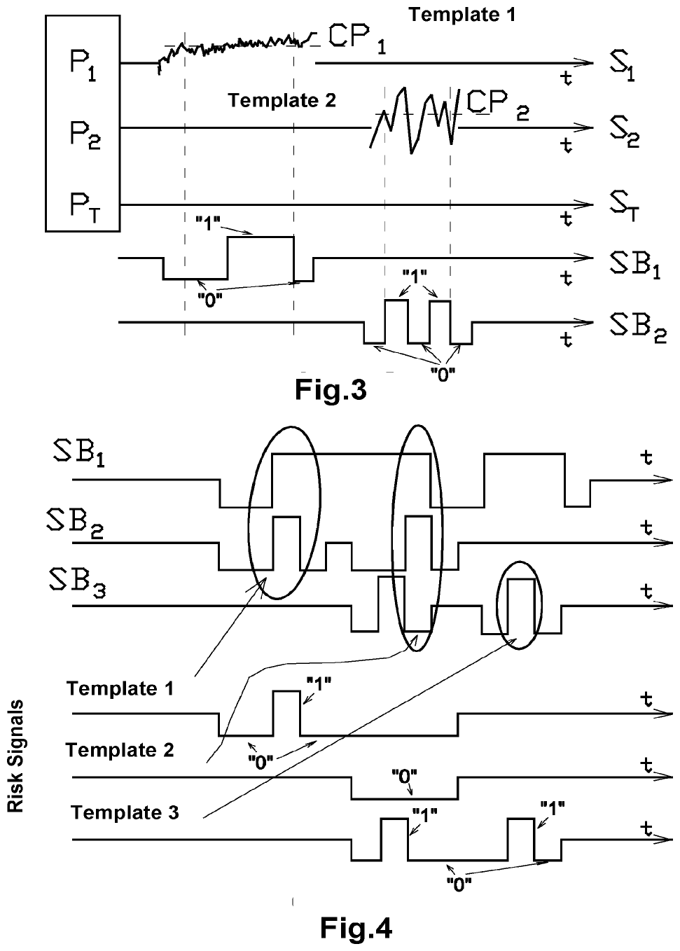

FIG. 3 shows an example of such conversion for conditional signals 51 and S2. Thus, for signal 51, critical value of the parameter is the threshold value of CP1, indicated by a dotted line, and for the signal S2 the threshold value of CP2. If this value at a given time interval is exceeded at the output of conversion block 6 «1» is recorded for signals CB1 or CB2, if not exceeded, then «0» is recorded.

One more thing should be noted. The method provides for possible averaging at time intervals of input signals S for tuning against interference. In addition, the signals S, containing the measured functional parameters may be absent, for example, due to the switched off wearable personal device, the presence of interference in the signal transmission and other objective reasons. In this case, no binary CB signals are formed after the conversion. This is illustrated on FIG. 3.

The next step (FIG. 1) includes comparison of binary signals within the templates in the signal comparison block 7. At the input of this conversion, a single stream of binary signals is formed. Thus, this conversion allows further comparison in terms of template criteria of signals that could not be compared before the conversion to binary form.

In this example we are talking about a comparison in the simplest binary form, i.e. «1» and «0». However, it may be possible to store the threshold excess in the previous step in the form of more digits, i.e. to store the value of the excess value. This makes it possible to take into account the value of exceeding the threshold of the critical signal value and the duration of such an exceedance when making a decision about the presence of a health risk.

Within the signal set of each of the prepared templates 4, there is a comparing the binary signals with each other and when the “1” values of the signals in the set temporarily coincide, a decision is made about the presence of certain health risks. This operation is illustrated on FIG. 4. Thus, binary signals SB₁, SB₂, SB₃ in this example are compared by “AND” logic within each of the templates: Template 1, Template 2, and Template 3. If at the time interval of comparison within the template each SB signal has a value «1», then the output will be «1». If there is even one «0», the output will be «0». In this example, when comparing the first template and the third template, the output contains «1», indicating that there is some health risk. If more than one templates are triggered at the same time, multiple health risks are identified.

As an example of health risk assessment, here is an example with signals from a wearable personal device containing temporal functional parameters of stress and hydration level measured in an observed man of 60 years of age. At the first phase of conversion into binary signals, both signal with the stress parameter and the signal with the parameter of hydration level (dehydration) are compared with the signals of the critical value threshold and the time of this exceeding. At the next phase, the signals of the binary form are already compared, within the framework of the corresponding templates. The following situations can be identified:
increased stress with low hydration;
prolonged low hydration (dehydration).

Health monitoring showed that a patient was chronically dehydrated. An appointment with the physician confirmed that after replacing one of the heart valves with an artificial heart valve 10 years ago, blood pressure lowering medications, which included a diuretic, had been taken for over the past two years, resulting in «blood clotting» caused by a condition of dehydration. At the same time, low hydration was accompanied by increased stress. In this example, the risk identified by the system was recognized by a physician as significant to the life and health of the person being observed and a new treatment was prescribed.

All of these situations suggest that there is an objective possibility of identifying risks to human health.

Based on these data, more general assessments of human health risks can be further considered. For example, these specified risks may indicate to cardiovascular disease, or metabolic disorders. In addition, evidence of stress-related risks and low hydration may indicate to decreased adaptive capacity or reduced performance.

An overall risk assessment for human health (FIG. 1, overall risk assessment block 8) can be built as a representation of risks in the form of a list of risks and their parameters, which will then be analyzed by specialists who make general health and overall risk decisions for the individual. An automated system can also be built, which will determine more general risks based on the data received, for all or part of the risk data received.

### INDUSTRIAL APPLICABILITY

The advantage of the method is the simplicity of implementation and versatility, allowing the assessment of health risks using any signals with any parameter data and data about the state of the human body.

1. A method for assessing human health risks based on measured functional parameters from a wearable personal device, the method comprising:
preliminary preparing a series of templates, wherein each template comprises a set of interrelated critical parameter values and temporal characteristics thereof in terms of duration and periodicity,
receiving signals comprising measured functional parameters from at least one wearable device, wherein each of the received signals is converted into a binary signal at a given time interval, wherein the signal is given a value of 1 if the signal exceeds a threshold of a critical parameter value which is stored in one of the plurality of pre-prepared templates, and a value of 0 if the signal does not exceed the threshold, and
comparing the binary signals with each other and, if the values of 1 temporally coincide among the set of signals of each of the pre-prepared template, a decision is made about the presence of certain health risks.

2. The method of claim 1, wherein the templates are preliminarily prepared for functional parameters received from wearable personal devices.

3. The method of claim 1, wherein each template comprises at least two parameters from the parameters obtained from wearable personal devices.

4. The method of claim 1, wherein signals received from the wearable personal device are signals comprising at least one parameter selected form the group consisting of a heart rate, sleep or wakefulness state, type of human physical activity, energy expenditure and inflow, body hydration state, sleep phases, and stress level.

5. The method of claim 1, wherein before converting signals from wearable personal devices into binary signals, an average value of the signal from the wearable personal device at a given time interval is determined.

6. The method of claim 1, wherein after converting each of the received signals into a binary signal, a single stream is formed from the binary signals.

7. The method of claim 1, wherein when a signal from the wearable personal device exceeds the threshold of a critical parameter value, a value of an excess and a duration of the excess are stored.

8. The method of claim 7, wherein when deciding whether there is a health risk, a magnitude of the exceedance of the threshold of the critical signal value and a duration of the exceedance are taken into consideration.

9. The method of claim 1, comprising obtaining from a single signal received from the wearable personal device in the process of converting it into a binary signal as many binary signals of a given parameter as there are different critical values of this parameter in the templates.

10. The method of claim 1, wherein at least one time window is used for each template to correlate the incoming data characterizing them for each of the signals is correlated.

11. The method of claim 10, wherein a length of each time window is determined by a specific template.

12. The method of claim 1, wherein an overall assessment of human health risk is performed based on health risk signals.
